(19) United States
(12) Patent Application Publication
(10) Pub. No.: US 2008/0038218 A1
(43) Pub. Date: Feb. 14, 2008

(54) Cosmetic composition comprising at least one electrophilic monomer, at least one fluoro compound and at least one liquid organic solvent

(76) Inventors: Gaelle Brun, Paris (FR); Aude Livoreil, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/784,000

(22) Filed: Apr. 13, 2007

Related U.S. Application Data
(60) Provisional application No. 60/796,218, filed on May 1, 2006.

(30) Foreign Application Priority Data
Apr. 13, 2006 (FR) 06/03281

(51) Int. Cl. A61K 8/72; A61Q 5/00

(52) U.S. Cl. 424/70.16; 424/70.11

(57) ABSTRACT

The present disclosure relates to a cosmetic composition for treating keratin fibers, for instance human keratin fibers such as the hair, comprising at least one liquid organic solvent, at least one electrophilic monomer and at least one organofluorine compound comprising at least 4 carbon atoms, with the exception of polytetrafluoroethylene, and pigments coated with fluoro compounds.

This application claims benefit of U.S. Provisional Application No. 60/796,218, filed May 1, 2006, the contents of which are incorporated herein by reference. This application also claims benefit of priority under 35 U.S.C. § 119 to French Patent Application No. FR 06/03281, filed Apr. 13, 2006, the contents of which are also incorporated herein by reference.

The present disclosure relates to a cosmetic composition for treating keratin fibers, for instance human keratin fibers such as the hair, comprising at least one electrophilic monomer, at least one particular organofluorine compound and at least one liquid organic solvent, to a use of this composition for treating the hair and also to a treatment process involving the composition as disclosed herein.

Hair is generally damaged and embrittled by the action of external atmospheric agents such as the light and bad weather, and by mechanical or chemical treatments such as blow-drying, combing, bleaching, permanent-waving and/or dyeing. As a result, the hair is often difficult to manage, for instance it becomes difficult to disentangle or style. A head of hair, even of lush hair, has difficulty in maintaining an attractive style due to the fact that the hair can lack vigor, volume and liveliness.

Thus, in order to overcome this, it is common practice to use styling products that allow the hair to be conditioned, for example, by giving it body, mass or volume.

These styling products are generally cosmetic hair compositions comprising at least one polymer with high affinity for the hair, which usually have the function of forming a film on the hair surface in order to modify its surface properties, such as to condition it.

One drawback associated with the use of these hair compositions lies in the fact that the cosmetic effects imparted by such compositions have a tendency to disappear, for instance, after the first shampoo wash.

In order to overcome this drawback, it may be envisaged to increase the remanence of the deposit of polymers by directly performing free-radical polymerization of certain monomers on the hair. However, the treatments used thus far tend to result in the degradation of the fiber and the hair treated in such a manner is generally difficult to disentangle.

Moreover, it is known practice, for example from French Patent No. FR 2 833 489, to use anionic-polymerizing electrophilic monomers directly at the surface of the hair in the presence of a nucleophilic agent such as hydroxide ions (OH⁻) contained in water at neutral pH. Thus, once applied to the hair, these monomers form a polymer that leads to a coating. However, the coating obtained using these compositions does not show satisfactory resistance relative to the various attacking factors to which the hair may be subjected.

There is thus a real need to find cosmetic compositions, for instance to condition the hair, that are remanent to shampooing and to external attack, while at the same time maintain good cosmetic properties, such as providing long-lasting body, mass or volume to the hair.

Thus, one aspect of the present disclosure is a cosmetic composition for treating keratin fibers, for instance human keratin fibers such as the hair, comprising:
at least one liquid organic solvent,
at least one electrophilic monomer, and
at least one organofluorine compound comprising at least 4 carbon atoms, with the exception of polytetrafluoroethylene, and pigments coated with fluoro compounds.

By applying such a composition, remanent coating can be obtained, which can have, for example, good resistance to shampooing and to external attack such as that caused by perspiration, light and fatty substances such as sebum. Moreover, the composition as disclosed herein affords a long-lasting improvement in the body, mass and volume of the hair.

The coating can be in the form of a smooth, uniform deposit that shows excellent adhesion to the hair.

As disclosed herein, the at least one electrophilic monomer, the at least one liquid organic solvent and the at least one fluoro compound are different compounds.

Another aspect of the present disclosure relates to a cosmetic process for treating keratin fibers using the cosmetic composition as disclosed herein.

The present disclosure also relates to the use of the cosmetic composition for treating keratin fibers.

A further aspect of the present disclosure is a multi-compartment device or kit comprising, in at least one first compartment, a composition (C) comprising at least one electrophilic monomer and, in at least one second compartment, a composition (D) comprising at least one organofluorine compound comprising at least 4 carbon atoms, with the exception of polytetrafluoroethylene, and pigments coated with fluoro compounds, wherein one and/or the other of composition (C) and composition (D) further comprising at least one liquid organic solvent.

Other subjects, characteristics, aspects and benefits of the present disclosure will emerge even more clearly upon reading the description and the examples that follow.

The at least one electrophilic monomer present in the composition of the present disclosure can be chosen from:
the benzylidene malononitrile derivatives (A), 2-(4-chloro-benzylidene)malononitrile (A1), ethyl 2-cyano-3-phenylacrylate (B), and ethyl 2-cyano-3-(4-chlorophenyl)acrylate (B1) described in Sayyah, J. Polymer Research, 2000, p. 97: 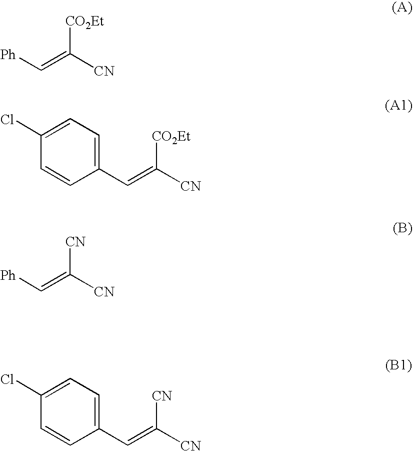
methylidenemalonate derivatives, for instance:
diethyl 2-methylenemalonate (C) by Hopff, Makromoleculare Chemie [Macromolecular Chemistry], 1961, p. 95, De Keyser, J. Pharm. Sci, 1991, p. 67 and Klemarczyk, Polymer, 1998, p. 173: 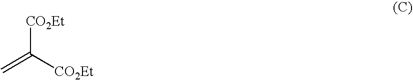
ethyl 2-ethoxycarbonylmethylenecarbonylacrylate (D) by Breton, Biomaterials, 1998, p. 271 and Couvreur, Pharmaceutical Research, 1994, p. 1270: 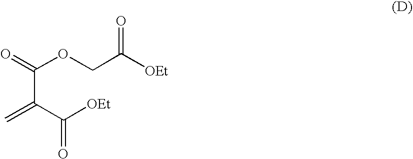
itaconate and itaconimide derivatives, for instance:
dimethyl itaconate (E) by Bachrach, European Polymer Journal, 1976, p. 563: 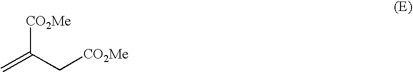
N-butyl itaconimide (F), N-(4-tolyl) itaconimide (G), N-(2-ethylphenyl) itaconimide (H), N-(2,6-diethylphenyl) itaconimide (I) by Wanatabe, J. Polymer Science: Part A: Polymer chemistry, 1994, p. 2073:

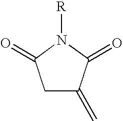

R=Bu (F), 4-tolyl (G), 2-ethylphenyl (H), 2,6-diethylphenyl (I)
the derivatives methyl α-(methylsulfonyl)acrylate (K), ethyl α-(methylsulfonyl)-acrylate (L), methyl α-(tert-butylsulfonyl)acrylate (M), tert-butyl α-(methylsulfonyl)acrylate (N) and tert-butyl α-(tert-butylsulfonyl)acrylate (O), by Gipstein, J. Org. Chem, 1980, p. 1486 and

the derivatives 1,1-bis(methylsulfonyl)ethylene (P), 1-acetyl-1-methylsulfonylethylene (Q), methyl α-(methylsulfonyl)vinylsulfonate (R) and a-methylsulfonylacrylonitrile (S), as described in U. S. Pat. No. 2,748,050: 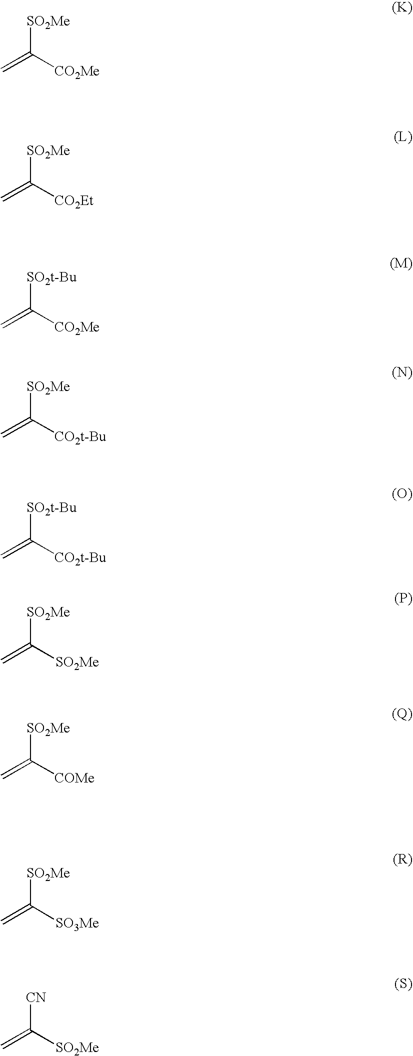
the methyl vinyl sulfone (T) and phenyl vinyl sulfone (U) derivatives by Boor, J. Polymer Science, 1971, p. 249: 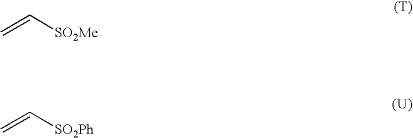
the phenyl vinyl sulfoxide derivative (V) by Kanga, Polymer preprints (ACS, Division of Polymer Chemistry), 1987, p. 322: 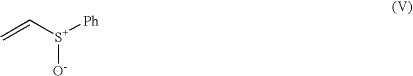
the derivative 3-methyl-N-(phenylsulfonyl)-1-aza-1,3-butadiene (W) by Bonner, Polymer Bulletin, 1992, p. 517: 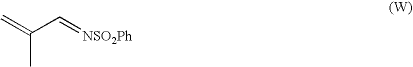
acrylate and acrylamide derivatives, for instance:
N-propyl-N-(3-triisopropoxysilylpropyl)acrylamide (X) and N-propyl-N-(3-triethoxysilylpropyl)acrylamide (Y) by Kobayashi, Journal of Polymer Science, Part A: Polymer Chemistry, 2005, p. 2754: 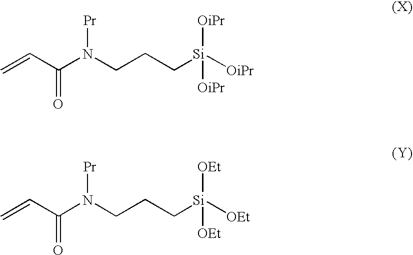
2-hydroxyethyl acrylate (Z) and 2-hydroxyethyl methacrylate (AA) by Rozenberg, International Journal of Plastics Technology, 2003, p. 17: 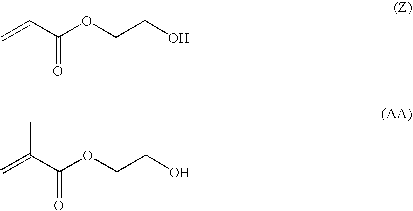
N-butyl acrylate (AB) described in Schmitt, Macromolecules, 2001, p. 2115, and
tert-butyl acrylate (AC) described in Ishizone, Macromolecules, 1999, p. 955:

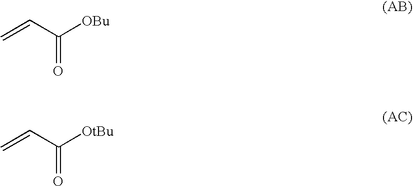

The electron-withdrawing monomer that is useful in the present disclosure may be cyclic or linear. When it is cyclic, the electron-withdrawing group can be exocyclic, for example. In other words, it does not form an integral part of the cyclic structure of the monomer.

According to at least one embodiment, these monomers may comprise at least two electron-withdrawing groups.

By way of non-limiting example, the monomers comprising at least two electron-withdrawing groups, may be made of the monomers of formula (I):

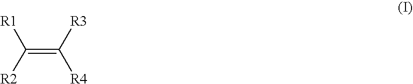

wherein:

R1 and R2 are, independently of each other, chosen from sparingly and non-electron-withdrawing groups (sparingly or non-inductive-withdrawing) such as:
a hydrogen atom,
a saturated or unsaturated, linear, branched or cyclic hydrocarbon-based group, for example containing from 1 to 20 carbon atoms, and further still from 1 to 10 carbon atoms, and optionally comprising at least one entity chosen from nitrogen, oxygen or sulfur, and optionally substituted with at least one group chosen from —OR, —COOR, —COR, —SH, —SR and —OH, and halogens,
a modified or unmodified polyorganosiloxane residue,
a polyoxyalkylene group,

R3 and R4 are, independently of each other, an electron-withdrawing (or inductive-withdrawing) group that, according to at least one embodiment, may be chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear and branched alkenyl groups, linear and branched alkynyl groups, C₁-C₄ mono- or polyfluoroalkyl groups, aryl groups such as phenyl, and aryloxy groups such as phenoxyloxy,

R is chosen from saturated and unsaturated, linear, branched and cyclic hydrocarbon-based groups, for instance containing from 1 to 20 carbon atoms and further, for example, from 1 to 10 carbon atoms, and optionally comprising at least one entity chosen from nitrogen, oxygen and sulfur, and optionally substituted with at least one entity chosen from —OR′, —COOR′, —COR′, —SH, —SR′, —OH, halogens, and polymer residues, this polymer possibly being obtained by free-radical polymerization, by polycondensation or by ring opening, where R′ is a C₁-C₁₀ alkyl group.

As used herein, the term “electron-withdrawing or inductive-withdrawing group (−I)” means any group that is more electronegative than carbon. For instance, reference may be made to the publication P. R. Wells, Prog. Phys. Org. Chem., Vol. 6, p. 111 (1968).

As used herein, the term “sparingly or non-electron-withdrawing group” mean any group whose electronegativity is less than or equal to that of carbon.

The alkenyl or alkynyl groups may contain from 2 to 20 carbon atoms and further still, for example, from 2 to 10 carbon atoms.

As saturated or unsaturated, linear, branched or cyclic hydrocarbon-based groups may contain from 1 to 20 carbon atoms, for example, non-limiting mention may be made of linear or branched alkyl, alkenyl or alkynyl groups, such as methyl, ethyl, n-butyl, tert-butyl, isobutyl, pentyl, hexyl, octyl, butenyl or butynyl; cycloalkyl or aromatic groups.

Examples of substituted hydrocarbon-based groups that may be mentioned include but are not limited to hydroxyalkyl and polyhaloalkyl groups.

Examples of unmodified polyorganosiloxanes that may be mentioned include but are not limited to polyalkylsiloxanes such as polydimethylsiloxanes, polyarylsiloxanes such as polyphenylsiloxanes, and polyarylalkylsiloxanes such as polymethylphenylsiloxanes.

By way of example, the modified polyorganosiloxanes that may be mentioned include but are not limited to polydimethylsiloxanes containing polyoxyalkylene and/or siloxy and/or silanol and/or amine and/or imine and/or fluoroalkyl groups.

The polyoxyalkylene groups that may be mentioned include but are not limited to polyoxyethylene groups and polyoxypropylene groups, for instance those containing 1 to 200 oxyalkylene units.

Among the mono- or polyfluoroalkyl groups that may be mentioned include but are not limited to groups such as —(CH₂)ₙ—(CF₂)ₘ—CF₃ or —(CH₂)ₙ—(CF₂)ₘ—CHF₂ with n=1 to 20 and m=1 to 20.

The substituents R1 to R4 may optionally be substituted with a group having cosmetic activity. The groups with cosmetic activities that may be used include those obtained from groups having coloring, antioxidant, UV-screening and conditioning functions.

As examples of groups having a coloring function, non-limiting mention may be made of azo, quinone, methine, cyanomethine and triarylmethane groups.

As examples of groups having an antioxidant function, non-limiting mention may be made, for instance, of groups of butylhydroxyanisole (BHA), butylhydroxytoluene (BHT) or vitamin E type.

As examples of groups having a UV-screening function, non-limiting mention may be made, for instance, of groups of the benzophenone, cinnamate, benzoate, benzylidenecamphor and dibenzoylmethane type.

As examples of groups having a conditioning function, non-limiting mention may be made, for example of cationic groups and groups of fatty ester type.

Among the at least one electrophilic monomers mentioned above, according to at least one embodiment, the monomers of the cyanoacrylate family and the derivatives thereof of formula (II) may be used:

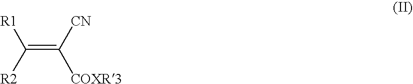

wherein:

X is chosen from NH, S and O,

R1 and R2 are defined as above, and according to at least one embodiment, R1 and R2 are hydrogen atoms,

R′3 is chosen from a hydrogen atom or R, as defined in formula (I).

According to another embodiment, X is O.

Compounds of formula (II) that may be mentioned include, but are not limited to the monomers:

a) belonging to the family of polyfluoroalkyl 2-cyanoacrylates such as the ester 2,2,3,3-tetrafluoropropyl 2-cyano-2-propenoate of formula (III):

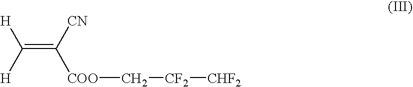

or the ester 2,2,2-trifluoroethyl 2-cyano-2-propenoate of formula (IV):

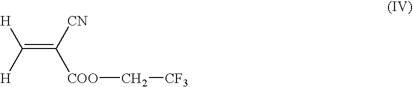

b) the alkyl or alkoxyalkyl 2-cyanoacrylates of formula (V):

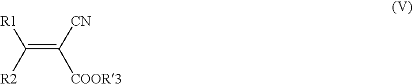

wherein:

R1 and R2 are defined as above,

R′3 is a C₁-C₁₀, (C₁-C₄)alkoxy(C₁-C₁₀)alkyl or C₂-C₁₀ alkenyl radical.

For example, non-limiting mention may be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

In the context of the present disclosure, the monomers defined in b) may be used. For example, according to one embodiment, the at least one electrophilic monomer is a cyanoacrylate monomer chosen from C₆-C₁₀ alkyl cyanoacrylates.

The monomers that may be used include but are not limited to the octyl cyanoacrylates of formula (VI) and mixtures thereof:

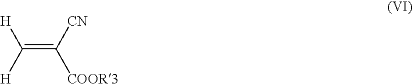

wherein R′3 is chosen from the following radicals:

—(CH₂)₇—CH₃;

—CH(CH₃)—(CH₂)₅—CH₃;

—CH₂—CH(C₂H₅)—(CH₂)₃—CH₃;

—(CH₂)₅—CH(CH₃)—CH₃; and

—(CH₂)₄—CH(C₂H₅)—CH₃.

The monomers used in accordance with the present disclosure may be covalently bonded to at least one support chosen from polymers, oligomers and dendrimers. The polymer or the oligomer may be linear, branched, in comb form or in block form. The distribution of the monomers of the present disclosure over the polymeric, oligomeric or dendritic structure may be random, in an end position or in the form of blocks.

According to at least one embodiment, the at least one electrophilic monomer is present in an amount ranging from 0.2% to 50% by weight, for example in an amount ranging from 0.5% to 30% by weight relative to the total weight of the cosmetic composition.

In the context of the present disclosure, the at least one electrophilic monomer of formula (I) may include monomers capable of undergoing anionic polymerization in the presence of at least one nucleophilic agent.

As used herein, the term “anionic polymerization” means the mechanism defined in the book “Advanced Organic Chemistry,” Third Edition by Jerry March, pages 151 to 161.

The nucleophilic agents capable of initiating the anionic polymerization are systems that are known per se, which are capable of generating a carbanion on contact with a nucleophilic agent, such as the hydroxide ions contained in water at neutral pH. The term “carbanion” refers to the chemical species defined in “Advanced Organic Chemistry,” Third Edition, by Jerry March, page 141.

The nucleophilic agents may be applied independent of the composition of the present disclosure. They may also be added to the composition at the time of use.

The at least one nucleophilic agent is a molecular compound, an oligomer, a dendrimer or a polymer containing nucleophilic functional groups. In a non-limiting manner, nucleophilic functional groups that may be mentioned include the following groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group; Ar is an aryl group, and R is a C₁-C₁₀ alkyl group.

The at least one electrophilic monomer of formula (I) as disclosed herein may be synthesized according to the known methods described in the art. For instance, the cyanoacrylate monomers may be synthesized according to the teaching of U.S. Pat. Nos. 3,527,224; 3,591,767; 3,667,472; 3,995,641; 4,035,334; and 4,650,826.

For the purposes of the present disclosure, the term “fluoro compound” means any organic compound comprising at least one fluorine atom, and comprising at least 4 carbon atoms. For instance, perfluoro compounds may be used, i.e. compounds for which the hydrogen atoms associated with at least one carbon atom are totally replaced with fluorine atoms.

This fluoro compound may be in the form of a defined compound or of a polymer, a silicone or a copolymer.

Such compounds are described, for example, in Ullmann's Industrial Chemistry Encyclopedia in the chapters on fluoro polymers (fluoroplastics and fluoroelastomers) and organic fluorine compounds.

According to at least one embodiment, the fluoro compound is not an acrylate-based grafted silicone polymer.

According to at least one embodiment, when the fluoro compound is liquid at 25° C. and atmospheric pressure, then the at least one liquid organic solvent is not fluorinated.

Among the fluoro compounds as defined herein, non-limiting mention may be made of:

i) fluorosilicone compounds of formula (VII):

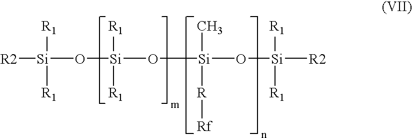

wherein:
R is chosen from C₁-C₆ linear and branched divalent alkyl groups such as divalent methyl, ethyl, propyl and butyl groups,
Rf is chosen from C₁-C₁₂fluoroalkyl radicals, such as a perfluoroalkyl radical, for example a C₁-C₉ fluoroalkyl radical,
R₁ is chosen from, independently of each other, C₁-C₂₀ alkyl radicals, hydroxyl radicals, and phenyl radicals,
R2 is R₁ or Rf,
m is chosen from 0 to 500, for example from 0 to 200, and
n is chosen from 1 to 1000, for example from 1 to 500.

According to at least one embodiment, the groups R₁ are identical and are methyl radicals.

Such compounds include those sold by the company Shin-Etsu under the names FL-5, FL-10, X22-821 and X22-822 or FL-100 by the company Dow Corning, under the name FS-1265 Fluid by the company Phoenix Chemical, under the name Pecosil FS or under the names Pecosil FSL-150, Pecosil FSL-300, Pecosil FSH-150, Pecosil FSH-300, Pecosil FSU-150 and Pecosil FSU-300.

ii) perfluorocycloalkyl compounds

For example, non-limiting mention may be made of the perfluorocycloalkyls chosen from those of formula (VIII):

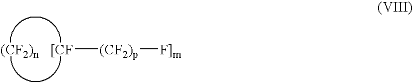

wherein n is equal to 3, 4 or 5, m is equal to 1 or 2, and p is equal to 0,1, 2 or 3; with the proviso that when m=2, the groups are not necessarily alpha to each other. The compounds of formula (VIII) that may be mentioned include but are not limited to perfluoromethylcyclopentane and 1,3-perfluorodimethylcyclohexane, sold, respectively, under the names Flutec PC1® and Flutec PC3® by the company F2 Chemicals, and also perfluorodimethylcyclobutane.

The perfluorocycloalkyl compounds that may also be mentioned include but are not limited to polycyclic compounds. For example, non-limiting mention may be made of perfluorodecalin and perfluoroperhydrophenanthrene, which are sold, respectively, under the names Flutec PC6® and Flutec PC11® by the company F2 Chemicals.

iii) the fluoroalkyl or heterofluoroalkyl compounds chosen from those of formula (IX):
CH₃—(CH₂)ₙ—[Z]ₜ-X—CF₃ (IX)

wherein:

t is 0 or 1;

n is 0, 1, 2 or 3;

X is chosen from linear and branched divalent perfluoroalkyl radicals containing from 2 to 5 carbon atoms; and

Z is chosen from O, S and NR; wherein

R is chosen from hydrogen, —(CH₂)ₙ—CH₃ and —(CF₂)ₘ—CF₃ radicals; and

m is 2, 3, 4 or 5.

Among the fluoroalkyl or heterofluoroalkyl compounds of formula (IX) that may be mentioned, by way of non-limiting example, are methoxynonafluorobutane sold under the name MSX 4518®, HFE-00® by the company 3M and ethoxynonafluorobutane sold under the name HFE-7200® by the company 3M.

iv) the perfluoroalkane compounds chosen from those of formula (X):
CF₃—(CF₂)ₙ—CF₃ (X)

wherein n ranges from 2 to 16, for example from 2 to 6.

The perfluoroalkane compounds of formula (X) that may be mentioned include but are not limited to perfluoro hexane, dodecafluoropentane and tetradecafluorohexane.

v) the perfluoropolyethers chosen from those of formulae (XI) and (XII):

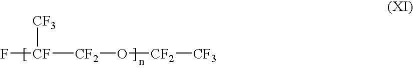

wherein n ranges from 7 to 30; and

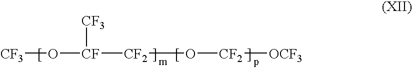

wherein the ratio m/p ranges from 20 to 40, and the molecular weight ranges from 500 to 20,000.

Among these perfluoropolyethers of formulae (XI) and (XII), non-limiting mention may be made, respectively, of the product sold under the name Fluortress LM36® by the company DuPont, and those sold under the general name Fomblin by the company Montefluos, for example Fomblin HC R®.

It is also possible to use the perfluoropolyethers mentioned in European Patent Application No. EP-A-641 194, the content of which is incorporated into the present disclosure by way of reference.

vi) the perfluoromorpholine derivatives chosen from those of formula (XIII):

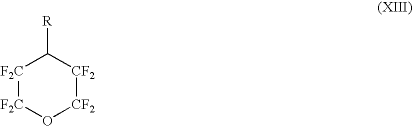

wherein R is a C₁-C₄ perfluoroalkyl radical.

The perfluoromorpholine derivatives of formula (XIII) that may be mentioned include but are not limited to 4-trifluoromethylperfluoromorpholine and 4-pentafluoroethylperfluoromorpholine.

vii) the fluoro esters chosen from those of formula (XIV):

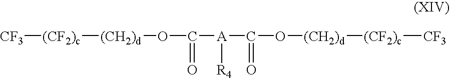

wherein:

R₄ is chosen from a hydrogen atom and a radical

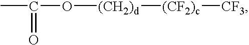

A is a linear or branched, optionally hydroxylated, C₁-C₁₈ alkylene or alkenylene chain,

c ranges from 1 to 17, and

d ranges from 1 to 18.

The compounds of formula (XIV) that may be mentioned include but are not limited to 2F-octylethyl-1,12-dodecanedioate of the following formula: 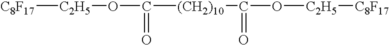
and C₁-C₆ perfluoroalkyl tricitrates, such as the perfluorobutyl tricitrate sold under the name Zonyl TBC® by the company DuPont.

viii) perfluoroalkylpolyacrylate-based copolymers.

For instance, according to at least one embodiment, the portion of the copolymer containing the perfluoroalkylpolyacrylate is formed by the polymerization of a monomer of acrylic type or of acrylamide type, such as those having the formula (A′):

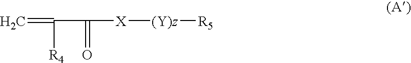

wherein:
R₄ is chosen from hydrogen and C₁-C₄ alkyl radicals,
X is chosen from O and —N—R₂, for which R₂ is chosen from hydrogen and C₁-C₁₀ alkyl and hydroxyalkyl radicals,
Y is chosen from C₁-C₆ alkylene radicals,
z is equal to 0 or 1,
R₅ is chosen from linear and branched C₁-C₂₀ alkyl radicals, all or some of the hydrogen atoms of which are replaced with fluorine atoms or a radical containing perfluoropolyether groups.

By way of example, the fluoro monomer may be chosen from those of formula:

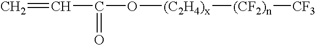

wherein:

n ranges from 4 to 16, where according to at least one embodiment, n is equal to 5, 7, 9 or 11; and

x is equal to 1 or 2.

The fluoro monomer may also be chosen from those of formula:

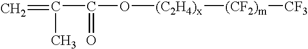

wherein:

m ranges from 4 to 16, where according to at least one embodiment, m is equal to 5, 7, 9 or 11; and

x is equal to 1 or 2.

The non-fluoro portion of the copolymer may be derived from monomers of the type B′ or C′ used alone or in combination, for example:

Monomer B′:

Alkyl (meth)acrylate-based monomer chosen from those of formula (B′):

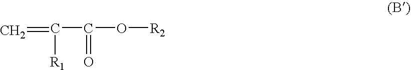

wherein:
R₁ is chosen from a hydrogen atom and C₁-C₄ alkyl radicals and
R₂ is chosen from C₁-C₂₀, such as C₂-C₈, alkyl radicals, C₂-C₆, such as C₂-C₄ hydroxycarbon radicals, and radicals —(CH₂)_{n—NH—R}₃ where R₃ is chosen from C₁-C₆ alkyl and cycloalkyl groups, and n is an integer that ranges from 1 to 4.

According to at least one embodiment, the radical R₁ of the monomer of formula B′ is chosen from hydrogen and methyl, ethyl, n-butyl and isopropyl radicals.

The monomers of formula B′ may be chosen from acrylic acid, methacrylic acid, methyl (meth)acrylate, ethyl (meth)acrylate, butyl (meth)acrylate, isobutyl (meth)acrylate, 2-ethylhexyl (meth)acrylate, lauryl (meth)acrylate, hydroxyethyl (meth)acrylate and 2-hydroxypropyl (meth)acrylate, and mixtures thereof, for example.

As disclosed herein, the fluoro copolymers of the present disclosure may be manufactured as described in French Patent Nos. FR-A-2 175 332 and FR-A-2 540 131 and European Patent No. EP-A-206 671.

These copolymers are, for example, those sold by Cognis under the brand name Repellan, those sold by Mitsubishi Chemicals under the brand name Repearl, or those sold by Clariant under the brand name Cartafluor UH Liquid.

Monomer C′:

Monomer comprises an alkyl(meth)acrylamide unit chosen from those of formula C′:

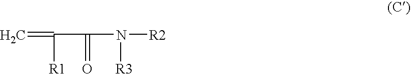

wherein R1 is H or CH₃;

R2 and R3, which may be identical or different, are chosen from H, linear and branched, saturated and unsaturated C₁-C₄₀ hydrocarbon-based radicals, optionally substituted with at least one group chosen from hydroxyl, carboxylic acid, sulfonic acid and phosphonic acid groups, which may or may not be esterified, C₅-C₁₀ cycloalkyl and C₅-C₁₀ aryl groups, optionally substituted with at least one group chosen from linear and branched C₁-C₄ alkyl, hydroxyl, carboxylic acid and sulfonic acid groups or possibly comprising, at its ends or within the group, at least one heteroatom chosen from O, S, N, Si and P, or a carbonyl group, or optionally comprising at least one heteroatom chosen from O, S and N.

By way of example, the (meth)acrylamide units that may be mentioned include but are not limited to N-tert-butylacrylamide, N-tert-hexylacrylamide, N-tert-octylacrylamide, N-octylacrylamide, N-decylacrylamide, N-dodecylacrylamide and N-methylundecylacrylamide.

ix) fluoro compound containing the following subunit:

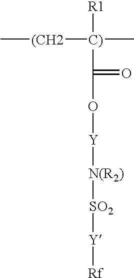

wherein:
R₁ is chosen from a hydrogen atom and C₁-C₄ alkyl radicals;
R₂ is chosen from a hydrogen atom and C₁-C₁₀ alkyl or hydroxyalkyl radicals;
Y and Y′ are C₁-C₆ alkylene radicals; and
Rf is chosen from linear and branched C₁-C₂₀ alkyl radicals, all or some of the hydrogen atoms of which are replaced with fluorine atoms or a radical containing perfluoropolyether groups.

For instance, the fluoro copolymers of the present disclosure may be synthesized as described in documents FR-A-2 175 332 and FR-A-2 540 131.

Examples of these copolymers may be found in the Scotchgard range sold by 3M, the formula of which is as follows: 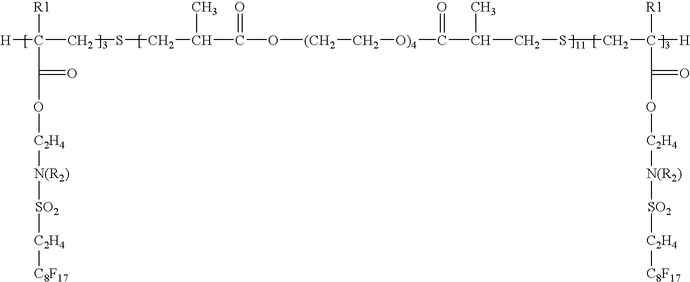
or in the Foraperle range sold by the company Atochem, the structure of which is as follows:

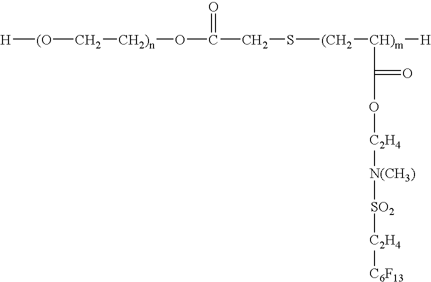

x) The fluoro compound may also be a copolymer based on fluoroethylene-alkyl vinyl ether. As a compound belonging to this family, non-limiting mention may be made of, for example, the Lumiflon range sold by Asahi Glass.

xi) The fluorohydrocarbons mentioned in Patent Application EP-A-609 132, the content of which is incorporated into the present disclosure by way of reference, may also be used as fluoro oils.

As a compound belonging to this family, non-limiting mention may be made of 1-(2′-fluorohexylethylthio)-3-(2″-ethylhexyloxy)-2-propanol.

By way of example, the fluoro compounds may be chosen from fluorosilicone compounds, perfluorocycloalkyls, perfluoropolyethers and perfluoroalkylpolyacrylate-based copolymers.

These fluoro compounds may be soluble or in dispersion in the medium of the composition. They may also be in the form of particles in dispersion, the dispersion optionally comprising at least one alcohol and/or ketone in low amount, this dispersion possibly being diluted in any proportion with water.

As disclosed herein, the fluoro compounds are present in the cosmetic composition in an amount generally ranging from 0.1% to 20% by weight, such as in an amount ranging from 0.2% to 15% by weight and further, for example, in an amount ranging from 0.5% to 10% by weight relative to the total weight of the said cosmetic composition.

As used herein, the term “organic solvent” means an organic substance capable of dissolving another substance without chemically modifying it.

The at least one liquid organic solvent that may prove useful in the cosmetic composition according to the present disclosure can be chosen from compounds that are liquid at a temperature of 25° C. and at 105 Pa (760 mmHg).

The at least one liquid organic solvent can be chosen, for example, from: aromatic alcohols such as benzyl alcohol; liquid fatty alcohols, for example C₁₀-C₃₀ aromatic alcohols; modified or unmodified polyols such as glycerol, glycol, propylene glycol, dipropylene glycol, butylene glycol or butyl diglycol; volatile silicones such as cyclopentasiloxane or cyclohexasiloxane; polydimethylsiloxanes optionally modified with alkyl and/or amine and/or imine and/or fluoroalkyl and/or carboxylic and/or betaine and/or quaternary ammonium functional groups, modified liquid polydimethylsiloxanes, mineral, organic and/or plant oils, alkanes, such as C₅-C₁₀ alkanes; and liquid fatty acids, liquid fatty esters and further for example, liquid fatty alcohol benzoates and salicylates.

The organic solvent may also be chosen from organic oils; silicones such as volatile silicones, amino or non-amino silicone gums or oils and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil or lemon oil, or alternatively organic compounds such as C₅-C₁₀ alkanes, acetone, methyl ethyl ketone, esters of liquid C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, esters of liquid C₁₀-C₃₀ fatty alcohols such as C₁₀-C₃₀ fatty alcohol benzoates, and mixtures thereof; polybutene oil, isononyl isononanoate, isostearyl malate, pentaerythrityl tetraisostearate, tridecyl trimellitate, and the mixture of cyclopentasiloxane (14.7% by weight)/polydimethylsiloxane dihydroxylated in the α and ω positions (85.3% by weight), and mixtures thereof. According to at least one embodiment, the at least one liquid organic solvent is not fluorinated.

According to at least one embodiment, the at least one liquid organic solvent comprises at least one silicone such as liquid polydimethylsiloxanes and modified liquid polydimethylsiloxanes, where their viscosity at 25° C. ranges from 0.1 cSt to 1,000,000 cSt, for instance it ranges from 1 cSt to 30,000 cSt.

Non-limiting mention may be made of the following oils:
the mixture of α,ω-dihydroxylated polydimethylsiloxane/cyclopentadimethylsiloxane (14.7/85.3) sold by Dow Corning under the name DC 1501 Fluid;
the mixture of α,ω-dihydroxylated polydimethylsiloxane/polydimethylsiloxane sold by Dow Corning under the name DC 1503 Fluid;
the mixture of dimethicone/cyclopentadimethylsiloxane sold by Dow Corning under the name DC 1411 Fluid or the product sold by Bayer under the name SF 1214;
the cyclopentadimethylsiloxane sold by Dow Corning under the name DC 245 Fluid;

and the respective mixtures of these oils.

The composition of the present disclosure can contain water. However, according to at least one embodiment, the composition as disclosed herein is anhydrous, i.e. it contains less than 1% by weight of water relative to the total weight of the composition.

The at least one liquid organic solvent of the composition is generally present in an amount ranging from 0.01% to 99%, for example from 50% to 99% by weight relative to the total weight of the composition.

The cosmetic composition according to the present disclosure may also comprise at least one pigment.

The use of at least one pigment in the cosmetic composition in accordance with the present disclosure makes it possible to obtain visible colorations even on dark hair, without making it necessary to bleach the keratin fibers, since the surface pigment masks the natural color of the fiber.

The composition in accordance with the present disclosure thus provides the benefit of producing colorations that show good resistance to the various attacking factors to which the hair may be subjected, such as fatty substances or successive shampooing over time.

Thus, it is possible to obtain visible and very chromatic colorations on a dark keratin fiber without it being necessary to lighten or bleach the keratin fibers and, consequently, without physical degradation of the keratin fibers.

For the purposes of the present disclosure, the term “pigment” means any organic and/or mineral species whose solubility in water is less than 0.01%, for instance less than 0.0001% at 20° C., having an absorption that ranges from 350 to 700 nm, for example at maximum absorption.

The at least one pigment that may be used can be chosen from organic and/or mineral pigments known in the art, for example those described in Kirk-Othmer's Encyclopedia of Chemical Technology and in Ullmann's Encyclopaedia of Industrial Chemistry.

These pigments may be in the form of powder or of pigmentary paste. The pigments may be coated or uncoated.

As disclosed herein, the pigments may be chosen, for example, from white or colored pigments, lakes, pigments with special effects such as nacres or flakes, and mixtures thereof.

Examples of white or colored mineral pigments that may be mentioned in a non-limiting manner include titanium dioxide, which may or may not be surface-treated, zirconium oxide or cerium oxide, iron oxide and chromium oxide, manganese violet, ultramarine blue, chromium hydrate and ferric blue. For example, the following mineral pigments may be used: Ta₂O₅, Ti₃O₅, Ti₂O₃, TiO, ZrO₂ as a mixture with TiO₂, ZrO₂, Nb₂O₅, CeO₂, ZnS.

Examples of white or colored organic pigments that may be mentioned include but are not limited to nitroso, nitro, azo, xanthene, quinoline, anthraquinone and phthalocyanin compounds, compounds of metallic complex type, and isoindolinone, isoindoline, quinacridone, perinone, perylene, diketopyrrolopyrrole, thioindigo, dioxazine, triphenylmethane and quinophthalone compounds.

For example, the white or colored organic pigments can be chosen from carmine, carbon black, aniline black, azo yellow, quinacridone, phthalocyanin blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in French Patent No. 2 679 771.

Pigmentary pastes of organic pigment may be used, such as the products sold by the company Hoechst under the names:
Jaune Cosmenyl IOG: Pigment Yellow 3 (CI 11710);
Jaune Cosmenyl G: Pigment Yellow 1 (CI 11680);
Orange Cosmenyl GR: Pigment Orange 43 (CI 71105);
Rouge Cosmenyl R: Pigment Red 4 (CI 12085);
Carmin Cosmenyl FB: Pigment Red 5 (CI 12490);
Violet Cosmenyl RL: Pigment Violet 23 (CI 51319);
Bleu Cosmenyl A2R: Pigment Blue 15.1 (CI 74160);
Vert Cosmenyl GG: Pigment Green 7 (CI 74260); and
Noir Cosmenyl R: Pigment Black 7 (CI 77266).

The pigments in accordance with the present disclosure may also be in the form of composite pigments as described in European Patent EP 1 184 426. These composite pigments may be composed of, for example, particles comprising a mineral core, at least one binder for fixing the organic pigments to the core, and at least one organic pigment at least partially covering the core.

As disclosed herein, the term “lakes” means dyes adsorbed onto insoluble particles, the assembly thus obtained remaining insoluble during use. The mineral substrates onto which the dyes are adsorbed are, for example, alumina, silica, calcium sodium borosilicate or calcium aluminium borosilicate, and aluminium. Among the organic dyes that may be used, non-limiting mention is made of cochineal carmine.

Examples of lakes that may be mentioned in a non-limiting manner include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), and D & C Blue 1 (CI 42 090).

As disclosed herein, the term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). They are consequently in contrast with white or colored pigments, which afford a standard opaque, semi-transparent or transparent uniform shade.

Examples of pigments with special effects that may be mentioned include white nacreous pigments such as mica coated with titanium or with bismuth oxychloride, colored nacreous pigments such as mica coated with titanium and with iron oxides, mica coated with titanium, such as with ferric blue or with chromium oxide, mica coated with titanium and with an organic pigment as defined above, and also nacreous pigments based on bismuth oxychloride. Nacreous pigments that may be mentioned include but are not limited to the Cellini nacre sold by Engelhard (Mica-TiO2-lake), the Prestige nacre sold by Eckart (Mica-TiO2), the Prestige Bronze nacre sold by Eckart (Mica-Fe2O3) and the Colorona nacre sold by Merck (Mica-TiO2-Fe2O3).

Non-limiting mention may also be made of pigments with an interference effect not bound to a substrate, for instance liquid crystals (Helicones HC from Wacker), holographic interference flakes (Geometric Pigments or Spectra f/x from Spectratek). The pigments with special effects also comprise fluorescent pigments, whether they are substances that are fluorescent in daylight or that produce ultraviolet fluorescence, phosphorescent pigments, photochromic pigments, thermochromic pigments and quantum dots, for example those sold by the company Quantum Dots Corporation.

Quantum dots are luminescent semiconductive nanoparticles capable of emitting, under light excitation, radiation with a wavelength in the range of 400 nm to 700 nm. These nanoparticles are known in the literature. For instance, they may be manufactured according to the processes described, for example, in U.S. Pat. Nos. 6,225,198 or 5,990,479, in the publications cited therein, and also in the following publications: Dabboussi B. O. et al “(CdSe)ZnS core-shell quantum dots: synthesis and characterisation of a size series of highly luminescent nanocristallites” Journal of Physical Chemistry B, Vol. 101, 1997, pp. 9463-9475 and Peng, Xiaogang et al, “Epitaxial Growth of highly Luminescent CdSe/CdS core/shell nanocrystals with photostability and electronic accessibility” Journal of the American Chemical Society, Vol. 119, No. 30, pp. 7019-7029.

The variety of pigments that may be used in the present disclosure makes it possible to obtain a rich palette of colors, for instance the possibility to obtain optical effects such as metallic or interference effects.

According to at least one embodiment, the pigments are colored pigments. The term “colored pigments” means pigments other than white pigments.

The size of the pigment used in the cosmetic composition according to the present disclosure generally ranges from 10 nm to 200 μm, for example from 20 nm to 80 μm and even further for example from 30 nm to 50 μm.

The pigments used in the cosmetic composition as disclosed herein may be surface-treated with an organic agent.

Thus, the pigments that have been surface-treated beforehand, which are useful in the context of the present disclosure, can be pigments that have totally or partially undergone a surface treatment of chemical, electronic, electrochemical, mechanochemical or mechanical nature, with an organic agent such as those described in Cosmetics and Toiletries, February 1990, Vol. 105, pp. 53-64, before being dispersed in the composition in accordance with the present disclosure. These organic agents may be chosen, for example, from amino acids; waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; and fluorosilicone compounds.

The surface-treated pigments that may prove useful in the cosmetic composition according to the present disclosure may also have been treated with a mixture of these compounds and/or may have undergone several surface treatments.

The surface-treated pigments that may prove useful in the context of the present disclosure may be prepared according to surface-treatment techniques that are well-known to those skilled in the art, or may be commercially available in the required form.

For example, the surface-treated pigments may be coated with an organic layer.

The organic agent with which the pigments are treated may be deposited on the pigments by evaporation of solvent, chemical reaction between the molecules of the surface agent or creation of a covalent bond between the surface agent and the pigments, for example.

The surface treatment may thus be performed, for example, by chemical reaction of a surface agent with the surface of the pigments and creation of a covalent bond between the surface agent and the pigments or the fillers. For instance, this method is described in U.S. Pat. No. 4,578,266.

According to at least one embodiment, an organic agent covalently bonded to the pigments will be used.

The agent for the surface treatment may be present in an amount ranging from 0.1% to 50% by weight, for instance from 0.5% to 30% by weight and further, for example from 1% to 10% by weight relative to the total weight of the surface-treated pigments.

The surface treatments of the pigments may be chosen from the following treatments, by way of non-limiting example:
a PEG-silicone treatment, for instance the AQ surface treatment sold by LCW;
a chitosan treatment, for instance the CTS surface treatment sold by LCW;
a triethoxycaprylylsilane treatment, for instance the AS surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a lauroyllysine treatment, for instance the LL surface treatment sold by LCW;
a lauroyllysine dimethicone treatment, for instance the LUSI surface treatment sold by LCW;
a magnesium myristate treatment, for instance the MM surface treatment sold by LCW;
an aluminium dimyristate treatment, for instance the MI surface treatment sold by Miyoshi;
an isostearyl sebacate treatment, for instance the HS surface treatment sold by Miyoshi;
a disodium stearoyl glutamate treatment, for instance the NAI surface treatment sold by Miyoshi;
a dimethicone/disodium stearoyl glutamate treatment, for instance the SA/NAI surface treatment sold by Miyoshi;
an acrylate/dimethicone copolymer and perfluoroalkyl phosphate treatment, for instance the FSA treatment sold by Daito;
a lauroyllysine/aluminium tristearate treatment, for instance the LL-AlSt surface treatment sold by Daito;
an octyltriethylsilane treatment, for instance the OTS surface treatment sold by Daito;
an octyltriethylsilane/perfluoroalkyl phosphate treatment, for instance the FOTS surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
a microcrystalline cellulose and carboxymethylcellulose treatment, for instance the AC surface treatment sold by Daito;
a cellulose treatment, for instance the C2 surface treatment sold by Daito; and
an acrylate copolymer treatment, for instance the APD surface treatment sold by Daito.

The at least one pigment may be generally present in the composition in total amounts generally ranging from 0.05% to 50% by weight, for example from 0.1% to 35% by weight and even further from 0.5% to 20% by weight relative to the total weight of the composition.

The composition in accordance with the present disclosure may further comprise at least one pigment that has not been surface-treated.

The medium of the composition of the present disclosure may also be in the form of an emulsion and/or may be encapsulated, the electrophilic monomers being maintained in an anhydrous medium until the time of use. When the medium is an emulsion, this emulsion comprises, for example, a dispersed or continuous phase that may comprise water, C₁-C₄ aliphatic alcohols and mixtures thereof and an anhydrous organic phase comprising the monomer. In the case of capsules or microcapsules, the capsule may comprise the monomer in an anhydrous medium and may be dispersed in an anhydrous medium as defined above, water, C₁-C₄ aliphatic alcohols, and mixtures thereof.

Polymerization inhibitors, such as anionic and/or free-radical polymerization inhibitors may also be introduced into the compositions, in order to enhance the stability of the composition over time. In a non-limiting manner, the following polymerization inhibitors may be mentioned: sulfur dioxide, nitric oxide, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole or hydroxyanisole, pyrogallol and derivatives thereof, p-methoxyphenol, hydroxybutyltoluene, alkyl sulfates, alkyl sulfites, alkyl sulfones, alkyl sulfoxides, alkyl sulfides, mercaptans and 3-sulfonene, and mixtures thereof. According to at least one embodiment, the alkyl groups contain 1 to 6 carbon atoms.

According to another embodiment, mineral or organic acids may also be used as an inhibitor.

Thus, the cosmetic composition as disclosed herein may also comprise at least one mineral or organic acid, where according to at least one embodiment, the acid comprises at least one carboxylic or sulfonic groups, with a pKa that ranges from 0 to 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid, trifluoroacetic acid, octanoic acid, heptanoic acid and hexanoic acid.

According to at least one embodiment, acetic acid is used.

The concentration of inhibitor in the cosmetic composition of the present disclosure may range from 10 ppm to 30% by weight for instance, from 10 ppm to 15% by weight relative to the total weight of the composition.

To modify the anionic polymerization kinetics, it is also possible to increase the nucleophilicity of the fiber via chemical conversion of the keratin material.

Examples that may be mentioned include but are not limited to the reduction of the disulfide bridges of which keratin is partly composed, into thiols, before applying the composition of the present disclosure. In a non-exhaustive manner, as reducing agents for the disulfide bridges of which keratin is partially composed, non-limiting mention may be made of the following compounds:
anhydrous sodium thiosulfate,
powdered sodium metabisulfite,
thiourea,
ammonium sulfite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulfoxylate,
isooctyl thioglycolate,
dl-cysteine, and
monoethanolamine thioglycolate.

The composition of the present disclosure may also comprise at least one polymer with no reactivity on the at least one cyanoacrylate monomer and which is capable of increasing the viscosity of the composition. Increasing the viscosity makes it possible to reduce the rate of polymerization of the cyanoacrylate monomers. To do this, it is possible to add to the composition of the present disclosure, in a non-exhaustive manner, polymethyl methacrylate (PMMA) or cyanoacrylate-based copolymers as described in U.S. Pat. No. 6,224,622.

The compositions in accordance with the present disclosure may also contain at least one agent usually used in cosmetics, for instance reducing agents, fatty substances, plasticizers, softeners, antifoams, moisturizers, pigments, clays, mineral fillers, UV-screening agents, mineral colloids, peptizers, solubilizing agents, fragrances, preserving agents, anionic, cationic, nonionic or amphoteric surfactants, fixing or non-fixing polymers, proteins, vitamins, direct dyes or oxidation dyes, nacreous agents, propellent gases, mineral or organic thickeners such as benzylidene sorbitol and N-acylamino acids, oxyethylenated or non-oxyethylenated waxes, paraffins, and C₁₀-C₃₀ fatty amides such as lauric diethanolamide, and mixtures thereof.

The compositions may be in various galenical forms such as a lotion, an aerosol mousse, a hair conditioner or a shampoo, a gel or a wax. The compositions may be contained, for example, in a pump-dispenser bottle or an aerosol spray. The compositions of the present disclosure, after application, may be rinsed out or left in.

When the composition is contained in an aerosol, it may comprise at least one propellant. The at least one propellant may be chosen from compressed or liquefied gases usually used for the preparation of aerosol compositions. Air, carbon dioxide, compressed nitrogen and a soluble gas such as dimethyl ether, halogenated or non- halogenated hydrocarbons (butane, propane or isobutane), and mixtures thereof may be used, for example.

According to the process of the present disclosure, the composition as disclosed herein may be applied to the keratin fibers, for instance human keratin fibers such as the hair, in the presence of at least one nucleophilic agent.

According to at least one embodiment of the process of the present disclosure, the at least one nucleophilic agent capable of initiating the polymerization of the electrophilic monomer may be applied beforehand to the keratin fibers. The at least one nucleophilic agent may be used pure, as a solution, in the form of an emulsion or may be encapsulated. It may also be added to the anhydrous composition at the time of use just before application to the keratin fibers.

According to at least one embodiment, the at least one nucleophilic agent is water. This water may be supplied, for example, by premoistening the keratin fibers. It may also be added directly to the composition before application.

According to another embodiment, it is possible to modify the polymerization kinetics by premoistening the fiber with an aqueous solution whose pH has been adjusted using a base, an acid or an acid/base mixture. The acid and/or the base may be mineral or organic.

According to one variant, the treatment process comprises a step of applying a composition (A) comprising at least one fluoro compound and optionally at least one nucleophilic agent, followed by a step of applying to the fibers at least one composition (B), comprising at least one electrophilic monomer, one and/or the other of the compositions containing at least one liquid organic solvent.

Alternatively, the at least one nucleophilic agent may also be in a separate composition.

According to yet another variant, the process for treating the keratin fibers may be a hair dyeing process, which may be performed in several steps: a first step that comprises applying a first composition comprising the at least one pigment to the fibers and a second step that comprises applying a second composition comprising, inter alia, at least one electrophilic monomer, at least one fluoro compound and at least one liquid organic solvent as defined above, the at least one nucleophilic agent being optionally present in the composition comprising the pigment or in a separate composition.

According to at least one embodiment, the cosmetic composition comprising the at least one pigment is an aqueous dispersion of pigments, which allows moistening of the fiber and initiation of the polymerization when the composition comprising the at least one electrophilic monomer, the at least one fluoro compound and the at least one liquid organic solvent is applied.

According to the process of the present disclosure, at least one embodiment comprises applying the at least one electrophilic monomer, the at least one fluoro compound and optionally the at least one pigment in the same composition.

The process of the present disclosure may comprise additional intermediate and/or final steps such as the application of a cosmetic product, a rinsing step and/or a drying step. The drying may be performed under a hood, with a hairdryer and/or with a smoothing iron. For instance, the application of the compositions in accordance with the present disclosure may be followed by rinsing.

It is also possible to perform multiple applications of the composition as disclosed herein in order to obtain a superposition of coats to achieve specific properties of the deposit in terms of chemical nature, mechanical strength, thickness, appearance or feel.

The present disclosure also relates to the use of a cosmetic composition as described above for treating the hair.

According to at least one embodiment, when the cosmetic composition does not comprise pigments, the cosmetic composition may be used for conditioning the hair.

According to another embodiment, the cosmetic composition comprises both the at least one fluoro compound and at least one pigment, thus the said composition may be used for coloring the hair and optionally for conditioning it.

Another aspect of the present disclosure is a multi-compartment device or kit, comprising at least one first compartment comprising a first composition (C) comprising the at least one electrophilic monomer as defined herein, and at least one second compartment comprising a second composition (D) comprising at least one organofluorine compound, with the exception of polytetrafluoroethylene, and pigments coated with fluoro compounds, one and/or the other of the compositions comprising at least one liquid organic solvent as defined above, one of the two, or both of the compositions possibly comprising at least one cosmetic additive as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

### Example 1

A styling composition according to the present disclosure was prepared from the following compounds:

| | | |
|---|---|---|
| α,ω-Dihydroxylated polydimethylsiloxane/ | 45 | g |
| cyclopentadimethylsiloxane (14.7/85.3) sold by Dow | | |
| Corning under the name DC 1501 Fluid | | |
| Cyclopentadimethylsiloxane sold by Dow Corning under | 42.75 | g |
| the name DC245 Fluid | | |
| Perfluoro polymethyl isopropyl ether sold by Solvay under | 2 | g |
| the name Fomblin HC 25 | | |
| Methylheptyl cyanoacrylate from Chemence | 10 | g |
| Acetic acid | 0.25 | g |

0.2 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 2

A styling composition according to the present disclosure was prepared from the following compounds:

| | | |
|---|---|---|
| DC 1501 Fluid | 45 | g |
| DC 245 Fluid | 42.75 | g |
| Perfluorophenanthrene sold by F2 Chemical under | 2 | g |
| the name Flutec PC11 | | |
| Methylheptyl cyanoacrylate from Chemence | 10 | g |
| Acetic acid | 0.25 | g |

0.2 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 3

A styling composition according to the present disclosure was prepared from the following compounds:

| | | |
|---|---|---|
| DC 1501 Fluid | 45 | g |
| DC 245 Fluid | 42.75 | g |
| Perfluorononyl dimethicone sold by Phoenix Chemical under | 2 | g |
| the name Pecosil FSL-150 | | |
| Methylheptyl cyanoacrylate from Chemence | 10 | g |
| Acetic acid | 0.25 | g |

0.2 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 4

A styling composition according to the present disclosure was prepared from the following compounds:

| | | |
|---|---|---|
| DC 245 Fluid | 84.75 | g |
| Perfluorononyl dimethicone sold by Phoenix Chemical under | 5 | g |
| the name Pecosil FSU-300 | | |
| Methylheptyl cyanoacrylate from Chemence | 10 | g |
| Acetic acid | 0.25 | g |

0.2 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 5

A styling composition according to the present disclosure was prepared from the following compounds:

| | | |
|---|---|---|
| DC 1501 Fluid | 45 | g |
| DC 245 Fluid | 42.75 | g |
| Trifluoropropyl dimethicone sold by Dow Corning under | 2 | g |
| the name FS-1265 Fluid | | |
| Methylheptyl cyanoacrylate from Chemence | 10 | g |
| Acetic acid | 0.25 | g |

0.2 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 6

Composition A was prepared from the following ingredients:

| | | |
|---|---|---|
| Cartafluor UH Liquid sold by Clariant | 10 | g |
| Water | 90 | g |

Composition B was prepared from the following ingredients:

| | | |
|---|---|---|
| DC 1501 Fluid | 45 | g |
| DC 245 Fluid | 44.75 | g |
| Methylheptyl cyanoacrylate from Chemence | 10 | g |
| Acetic acid | 0.25 | g |

0.3 g of composition (A) was applied to a lock of 1 g of clean, wet hair. 0.2 g of composition (B) was then applied to this lock. After a leave-on time of 15 minutes, the lock was dried with a hairdryer for 2 minutes. The resultant lock had hairs that were individualized with a pleasant, remanent texture.

### Example 7

Composition (A) was prepared from the following ingredients:

| | |
|---|---|
| Foraperle 321 sold by DuPont de Nemours | 20 g |
| Water | 80 g |

Composition (B) was prepared from the following ingredients:

| | | |
|---|---|---|
| DC 1501 Fluid | 45 | g |
| DC 245 Fluid | 44.75 | g |
| Methylheptyl cyanoacrylate from Chemence | 10 | g |
| Acetic acid | 0.25 | g |

0.3 g of composition (A) was applied to a lock of 1 g of clean, wet hair. 0.2 g of composition (B) was then applied to this lock. After a leave-on time of 15 minutes, the lock was dried with a hairdryer for 2 minutes. The resultant lock had hairs that were individualized with a pleasant, remanent texture.

### Example 8

A dye composition according to the present disclosure was prepared from the following compounds:

| | | |
|---|---|---|
| DC 1501 Fluid | 40 | g |
| DC 245 Fluid | 37.75 | g |
| Mica nacre coated with brown iron oxide, sold by Eckart | 10 | g |
| under the name Prestige Bronze | | |
| Perfluoro polymethyl isopropyl ether sold by Solvay under | 2 | g |
| the name Fomblin HC 25 | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

### Example 9

A dye composition according to the present disclosure was prepared from the following compounds:

| | | |
|---|---|---|
| DC 1501 Fluid | 40 | g |
| DC 245 Fluid | 37.75 | g |
| Mica nacre coated with brown iron oxide, sold by Eckart | 10 | g |
| under the name Prestige Bronze | | |
| Perfluoro polymethyl isopropyl ether sold by Solvay under | 2 | g |
| the name Fomblin HC 25 | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

### Example 10

A dye composition according to the present disclosure was prepared from the following compounds:

| | | |
|---|---|---|
| DC 1501 Fluid | 40 | g |
| DC 245 Fluid | 37.75 | g |
| Mica nacre coated with brown iron oxide, | 10 | g |
| sold by Eckart under the name Prestige Bronze | | |
| Perfluorophenanthrene sold by F2 Chemical | 2 | g |
| under the name Flutec PC11 | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

### Example 11

A dye composition according to the present disclosure was prepared from the following compounds:

| | | |
|---|---|---|
| DC 1501 Fluid | 40 | g |
| DC 245 Fluid | 37.75 | g |
| Mica nacre coated with brown iron oxide, sold by | 10 | g |
| Eckart under the name Prestige Bronze | | |
| Perfluorononyl dimethicone sold by Phoenix Chemical | 2 | g |
| under the name Pecosil FSL-150 | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

### Example 12

A dye composition according to the present disclosure was prepared from the following compounds:

| | | |
|---|---|---|
| DC 1501 Fluid | 40 | g |
| DC 245 Fluid | 37.75 | g |
| Mica nacre coated with brown iron oxide, sold by | 10 | g |
| Eckart under the name Prestige Bronze | | |
| Trifluoropropyl dimethicone sold by Dow Corning | 2 | g |
| under the name FS-1265 Fluid | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

### Example 13

Composition (A) was prepared from the following ingredients:

| | |
|---|---|
| Cartafluor UH Liquid sold by Clariant | 10 g |
| Water | 90 g |

Composition (B) was prepared from the following ingredients:

| | | |
|---|---|---|
| DC 1501 Fluid | 40 | g |
| DC 245 Fluid | 39.75 | g |
| Mica nacre coated with brown iron oxide, | 10 | g |
| sold by Eckart under the name Prestige Bronze | | |
| Methylheptyl cyanoacrylate from Chemence | 10 | g |
| Acetic acid | 0.25 | g |

0.3 g of composition A was applied to a lock of 1 g of clean, wet hair. 0.5 g of composition B was then applied to this lock. After a leave-on time of 15 minutes, the lock was dried with a hairdryer for 2 minutes. The resultant lock had hairs that were individualized with a pleasant, remanent texture.

What is claimed is:

1. A cosmetic composition for treating keratin fibers, comprising:
at least one liquid organic solvent;
at least one electrophilic monomer; and
at least one organofluorine compound comprising at least 4 carbon atoms, with the exception of polytetrafluoroethylene, and pigments coated with fluoro compounds.

2. The cosmetic composition according to claim 1, wherein the at least one electrophilic monomer is chosen from those of formula (I): 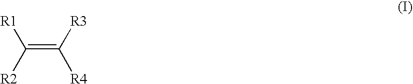
wherein:
R1 and R2 are chosen from, independently of each other, sparingly or non- electron-withdrawing groups, and
R3 and R4 are chosen from, independently of each other, electron-withdrawing groups.

3. The cosmetic composition according to claim 2, wherein the at least one electrophilic monomer of formula (I) is such that:
R1 and R2, independently of each other, are chosen from hydrogen, saturated and unsaturated, linear, branched and cyclic hydrocarbon-based groups that optionally comprise at least one entity chosen from nitrogen, oxygen and sulfur, and are optionally substituted with at least one entity chosen from —OR, —COOR, —COR, —SH, —SR, —OH, and halogens;
R is chosen from saturated and unsaturated, linear, branched and cyclic hydrocarbon-based groups that optionally comprise at least one entity chosen from nitrogen, oxygen and sulfur, and is optionally substituted with at least one entity chosen from —OR′, —COOR′, —COR′, —SH, —SR′ and —OH, halogens, or a polymer residue; and
R′ is a C₁-C₁₀ alkyl radical.

4. The cosmetic composition according to claim 2, wherein the at least one electrophilic monomer of formula (I) is such that:
R3 and R4, independently of each other, are chosen from: —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear and branched alkenyl groups, linear and branched alkynyl groups, C₁-C₄ mono- and/or polyfluoroalkyl groups, and aryl and/or aryloxy groups;
R is chosen from saturated and unsaturated, linear, branched and cyclic hydrocarbon-based groups that optionally comprises at least one entity chosen from nitrogen, oxygen and sulfur, and is optionally substituted with at least one entity chosen from —OR′, —COOR′, —COR′, —SH, —SR′, —OH, halogens, and polymer residues; and
R′ is a C₁-C₁₀ alkyl radical.

5. The cosmetic composition according to claim 1, wherein the at least one electrophilic monomer is a cyanoacrylate monomer chosen from those of formula (II): 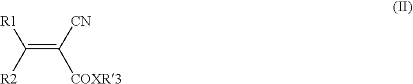
wherein:
X is chosen from NH, S and O;
R′3 is chosen from a hydrogen atom and R;
R is chosen from saturated and unsaturated, linear, branched and cyclic hydrocarbon-based groups that optionally comprise at least one entity chosen from nitrogen, oxygen and sulfur, and is optionally substituted with at least one entity chosen from —OR′, —COOR′, —COR′, —SH, —SR′, —OH, halogens, and polymer residues; and
R1 and R2, independently of each other, are chosen from a hydrogen atom, saturated and unsaturated, linear, branched and cyclic hydrocarbon-based groups that optionally comprise at least one entity chosen from nitrogen, oxygen and sulfur, and are optionally substituted with at least one entity chosen from —OR, —COOR, —COR, —SH, —SR, —OH, and halogens.

6. The cosmetic composition according to claim 2, wherein the at least one electrophilic monomer according to formula (I) is such that R1 and R2 are hydrogen atoms.

7. The cosmetic composition according to claim 1, wherein the at least one electrophilic monomer is a cyanoacrylate monomer chosen from those of formula (V): 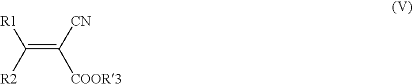
wherein:
R′3 is chosen from C₁-C₁₀ alkyl, (C₁-C₄)alkoxy(C₁-C₁₀)alkyl and C₂-C₁₀ alkenyl radicals; and
R1 and R2, independently of each other, are chosen from a hydrogen atom, saturated and unsaturated, linear, branched and cyclic hydrocarbon-based groups that optionally comprise at least one entity chosen from nitrogen, oxygen and sulfur, and are optionally substituted with at least one entity chosen from —OR, —COOR, —COR, —SH, —SR, —OH, and halogens.

8. The cosmetic composition according to claim 7, wherein R′3 is chosen from C₆-C₁₀ alkyl radicals.

9. The cosmetic composition according to claim 7, wherein R1 and R2 are hydrogen atoms.

10. The cosmetic composition according to claim 1, wherein the at least one electrophilic monomer is an alkyl cyanoacrylate chosen from those of formula (VI): 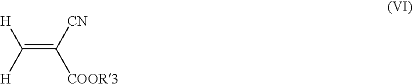
wherein:
R′3 is chosen from:
—(CH₂)₇—CH₃;
—CH(CH₃)—(CH₂)₅—CH₃;
—CH₂—CH(C₂H₅)—(CH₂)₃—CH₃;
—(CH₂)₅—CH(CH₃)—CH₃; and
—(CH₂)₄—CH(C₂H₅)—CH₃.

11. The cosmetic composition according to claim 1, wherein the at least one electrophilic monomer is a cyanoacrylate monomer and is covalently bonded to at least one support chosen from polymers, oligomers and dendrimers.

12. The cosmetic composition according to claim 1, wherein the at least one fluoro compound is a fluorosilicone compound chosen from those of formula (VII): 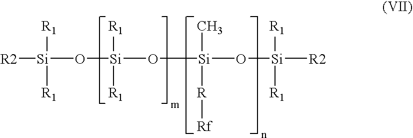
wherein:
R is chosen from C₁-C₆ linear and branched divalent alkyl groups;
Rf is chosen from fluoroalkyl radicals;
R₁ is chosen from, independently of each other, C₁-C₂₀ alkyl radicals, hydroxyl radicals, and phenyl radicals;
R2 is R₁ or Rf;
m is chosen from 0 to 500; and
n is chosen from 1 to 1000.

13. The cosmetic composition according to claim 12, wherein the groups R₁ are identical and are methyl radicals.

14. The cosmetic composition according to claim 1, wherein the at least one fluoro compound is chosen from the perfluorocycloalkyls of formula (VIII): 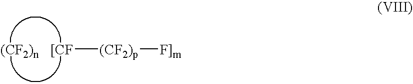
wherein:
n is equal to 3, 4 or 5;
m is equal to 1 or 2; and
p is equal to 0, 1, 2 or 3;
with the proviso that when m=2, the groups are not necessarily alpha to each other.

15. The cosmetic composition according to claim 1, wherein the at least one fluoro compound is chosen from the fluoroalkyl or heterofluoroalkyl compounds chosen from those of formula (IX):
CH₃—(CH₂)ₙ—[Z]ₜ-X—CF₃ (IX)
wherein:
t is 0 or 1;
n is 0, 1, 2 or 3;
X is a linear or branched divalent perfluoroalkyl radical containing from 2 to 5 carbon atoms; and
Z is chosen from O, S and NR, where R is chosen from a hydrogen, —(CH₂)ₙ—CH₃ and —(CF₂)ₘ—CF₃ radicals, where m is 2, 3, 4 or 5.

16. The cosmetic composition according to claim 1, wherein the at least one fluoro compound is a perfluoroalkane chosen from those of formula (X):
CF₃—(CF₂)ₙ—CF₃ (X)
where n ranges from 2 to 16.

17. The cosmetic composition according to claim 1, wherein the at least one fluoro compound is chosen from the perfluoropolyethers of formulae (XI) and (XII): 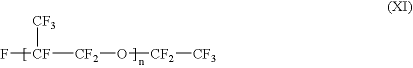
where n ranges from 7 to 30; and 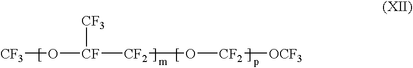
where the ratio m/p ranges from 20 to 40, and the molecular weight ranges from 500 to 20,000.

18. The cosmetic composition according to claim 1, wherein the at least one fluoro compound is chosen from the perfluoromorpholine derivatives of formula (XIII): 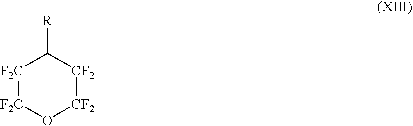
where R is chosen from C₁-C₄ perfluoroalkyl radicals.

19. The cosmetic composition according to claim 1, wherein the at least one fluoro compound is chosen from the fluoro esters of formula (XIV): 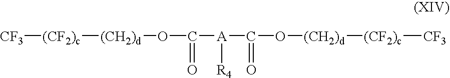
wherein:
R₄ is a hydrogen atom or a radical 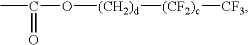
A is chosen from linear and branched, optionally hydroxylated, C₁-C₁₈ alkylene and alkenylene chains,
c is 1 to 17, and
d is 1 to 18.

20. The cosmetic composition according to claim 1, wherein the at least one fluoro compound is chosen from the perfluoroalkylpolyacrylate-based copolymers that can be obtained by polymerization using:
(i) a monomer of acrylic type or of acrylamide type chosen from those of formula (A′): 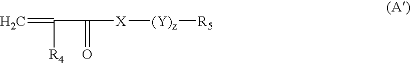
wherein:
R₄ is chosen from a hydrogen atom and C₁-C₄ alkyl radicals,
X is chosen from O and —N—R₂, where R₂ is chosen from a hydrogen atom and C₁-C₁₀ alkyl and hydroxyalkyl radicals,
Y is chosen from C₁-C₆ alkylene radicals,
z is equal to 0 or 1,
R₅ is chosen from linear and branched C₁-C₂₀ alkyl radicals, all or some of the hydrogen atoms of which are replaced with fluorine atoms, or a radical comprising perfluoropolyether groups;
(ii) a monomer of the type B′ chosen from those of formula (B′): 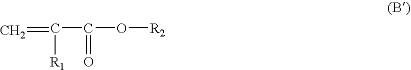
wherein:
R₁ is chosen from a hydrogen atom and C₁-C₄ alkyl radicals, and
R₂ is chosen from C₁-C₂₀ alkyl radicals, C₂-C₆ hydroxycarbon radicals, and —(CH₂)ₙ—NH—R₃ radicals where R₃ is chosen from C₁-C₆ alkyl and cycloalkyls and n is an integer ranging from 1 to 4;
(iii) a monomer of the type C′ comprising an alkyl(meth)acrylamide unit chosen from those of formula C′: 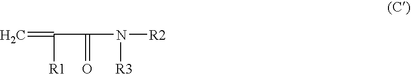
wherein:
R1 is chosen from H and CH₃;
R2 and R3, which may be identical or different, are chosen from H, linear and branched, saturated and unsaturated C₁-C₄₀ hydrocarbon-based radicals, optionally substituted with at least one group chosen from hydroxyl, carboxylic acid, sulfonic acid and phosphonic acid, which may or may not be esterified, C₅-C₁₀ cycloalkyl and C₅-C₁₀ aryl groups, optionally substituted with at least one group chosen from linear and branched C₁-C₄ alkyl, hydroxyl, carboxylic acid and sulfonic acid groups or optionally comprising, at its ends or within the group, at least one heteroatom chosen from O, S, N, Si and P or a carbonyl group, or optionally comprising at least one heteroatom chosen from O, S and N.

21. The cosmetic composition according to claim 1, wherein the at least one fluoro compound is chosen from the fluoro compounds comprising the following subunit: 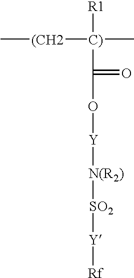
wherein:
R₁ is chosen from hydrogen and C₁-C₄ alkyl radicals,
R₂ is chosen from hydrogen and C₁-C₁₀ alkyl and/or hydroxyalkyl radicals,
Y and Y′ are chosen from C₁-C₆ alkylene radicals, and
Rf is chosen from linear and branched C₁-C₂₀ alkyl radicals, all or some of the hydrogen atoms of which are replaced with fluorine atoms or a radical comprising perfluoropolyether groups.

22. The cosmetic composition according to claim 1, wherein the at least one fluoro compound is chosen from fluorohydrocarbons.

23. The cosmetic composition according to claim 1, wherein the at least one fluoro compound is chosen from fluorosilicone compounds, perfluorocycloalkyls, perfluoropolyethers and perfluoroalkylpolyacrylate-based copolymers.

24. The cosmetic composition according to claim 1, wherein the at least one liquid organic solvent is chosen from aromatic alcohols; polyols; volatile silicones; polydimethylsiloxanes optionally modified with alkyl and/or amine and/or imine and/or fluoroalkyl and/or carboxylic and/or betaine and/or quaternary ammonium functional groups, modified liquid polydimethylsiloxanes, mineral, organic and/or plant oils, alkanes; liquid fatty acids and liquid fatty esters.

25. The cosmetic composition according to claim 24, wherein the at least one liquid organic solvent comprises a mixture of α,ω-dihydroxylated polydimethylsiloxane/cyclopentadimethylsiloxane and cyclopentadimethylsiloxane.

26. The cosmetic composition according to claim 24, wherein the at least one liquid organic solvent is present in an amount ranging from greater than 50% to 99% by weight relative to the total weight of the cosmetic composition.

27. The cosmetic composition according to claim 1, further comprising at least one organic pigment.

28. The cosmetic composition according to claim 1, wherein the composition is anhydrous.

29. The cosmetic composition according to claim 1, further comprising at least one nucleophilic agent.

30. The cosmetic composition according to claim 29, wherein the at least one nucleophilic agent is water.

31. A cosmetic process for treating keratin fibers, comprising:
applying a cosmetic composition to the keratin fibers in the presence of at least one nucleophilic agent, wherein the cosmetic composition comprises:
at least one liquid organic solvent;
at least one electrophilic monomer; and
at least one organofluorine compound comprising at least 4 carbon atoms, with the exception of polytetrafluoroethylene, and pigments coated with fluoro compounds.

32. The cosmetic process according to claim 31, wherein the cosmetic composition further comprises at least one nucleophilic agent.

33. The cosmetic process according to claim 32, wherein the at least one nucleophilic agent is water.

34. A cosmetic process comprising:
applying a composition (A) to the keratin fibers, where the composition (A) comprises at least one fluoro compound and optionally at least one nucleophilic agent; and
applying to the fibers at least one composition (B) comprising at least one electrophilic monomer,
wherein composition (A) and/or composition (B) further comprises at least one liquid organic solvent.

35. The cosmetic process according to claim 31, comprising:
applying the cosmetic composition to the keratin fibers, where the cosmetic composition further comprises at least one pigment; and
applying the cosmetic composition to the keratin fibers, where the cosmetic composition further comprises the at least one pigment and at least one nucleophilic agent.

36. The cosmetic process according to claim 31, comprising:
applying a first composition to the keratin fibers, where the first composition comprises the cosmetic composition and at least one pigment; and
applying a second composition to the keratin fibers, where the second composition comprises the cosmetic composition and a nucleophilic agent.

37. A method for providing a remanent coating on human keratin fibers comprising applying a cosmetic composition on human keratin fibers where the cosmetic composition comprises:
at least one liquid organic solvent;
at least one electrophilic monomer; and
at least one organofluorine compound comprising at least 4 carbon atoms, with the exception of polytetrafluoroethylene, and pigments coated with fluoro compounds.

38. A multi-compartment kit, comprising:
at least one first compartment comprising at least one first composition (C) comprising at least one electrophilic monomer, and
at least one second compartment comprising at least one second composition (D) comprising at least one organofluorine compound comprising at least 4 carbon atoms, with the exception of polytetrafluoroethylene, and pigments coated with fluoro compounds,
wherein at least one of composition (C) and/or composition (D) comprise at least one liquid organic solvent, and optionally at least one of composition (C) and/or composition (D) comprise at least one cosmetic additive.
